(19) United States
(12) Patent Application Publication
(10) Pub. No.: US 2024/0309362 A1
(43) Pub. Date: Sep. 19, 2024

(54) NON-RIBOSOMAL SEQUENCE ENRICHMENT AND SINGLE-STRANDED DNA LIBRARY FOR NUCLEIC ACID GUIDED NUCLEASE TARGETING

(71) Applicant: ARC BIO, LLC, Cambridge, MA (US)

(72) Inventors: Stephane B. GOURGUECHON, Cambridge, MA (US); Manuel Bernhard KRISPIN, Cambridge, MA (US)

(21) Appl. No.: 18/577,037

(22) PCT Filed: Jul. 7, 2022
(86) PCT No.: PCT/US2022/036368

§ 371 Date: Jan. 5, 2024

Related U.S. Application Data
(60) Provisional application No. 63/219,281, filed on Jul. 7, 2021.

(51) Int. Cl. C12N 15/10; C12N 9/22; C12N 15/11

(52) U.S. Cl. CPC C12N 15/1093; C12N 9/22; C12N 15/11; C12N 2310/20

(57) ABSTRACT

The present invention provides methods for selectively depleting unwanted sequences from a pool of nucleic acids, including (1) methods that can be used to deplete an unwanted sequence from a single-stranded DNA library, and (2) methods that can be used to deplete an unwanted non-polyadenylated RNA from a pool of single-stranded RNA molecules. By depleting the unwanted sequences, the methods enrich a sample for sequences of interest.

### CROSS-REFERENCE TO RELATED APPLICATIONS

This application claims priority to U.S. Provisional Application No. 63/219,281 filed on Jul. 7, 2021, the contents of which are incorporated by reference in their entireties

### BACKGROUND

Many sample libraries contain highly abundant sequences that have little informative value and increase the cost of sequencing. While methods such as hybridization capture have been developed to deplete these sequences, these methods are often time-consuming and can be inefficient. Moreover, hybridization capture is used to capture DNA sequences of interest while discarding the remaining sequences. As a result, hybridization capture is not a viable option when the sequences of interest are not known in advance. This is the case, for example, when one is interested in the microbial DNA sequences that are present in a host organism. DNA libraries derived from human tissues such as blood, vagina, nasal mucosal membrane, and lung typically contain >90% human and <10% microbial DNA. Thus, to detect a microbe in such tissues using shotgun sequencing, one would need to sequence to a very high coverage to ensure that the microbial DNA can be accurately identified. Achieving this depth of sequencing is expensive and, thus, untenable for many researchers. The same issue affects many other sequencing applications. For example, in cancer profiling, the mutant tumor-derived DNA sequences may be vastly outnumbered by wild-type DNA sequences due to the abundance of tumor-infiltrating immune cells or the interspersed nature of some tumors throughout normal tissue. Thus, there is a need in the art for methods that can be used to deplete specific unwanted sequences and, thereby, increase the percentage of sequences of interest within a library.

### SUMMARY

In a first aspect, the present invention provides methods for depleting an unwanted target sequence from a single-stranded DNA (ssDNA) library, thereby enriching for sequences of interest within the library. In these methods, the library comprises ssDNA molecules that each comprise a 5′ adapter and a 3′ adapter and are bound by single-stranded DNA-binding proteins (SSBs; i.e., to stabilize the ssDNA molecules against chemical attack). The unwanted target sequence is depleted using a nucleic acid-guided nuclease (e.g., Cas9). Since many nucleic acid-guided nucleases only cut double-stranded DNA (dsDNA), these methods involve converting the target sequence into dsDNA via hybridization to a complementary sequence prior to nuclease cleavage. These methods can be subdivided into three protocols, i.e., Protocol 1, Protocol 2, and Protocol 3.

In Protocol 1 (see FIG. 1, arrow 1), the method comprises: (a) contacting the library with proteinase K to degrade at least a portion of the SSBs; (b) contacting the library with a targeting oligonucleotide that is complementary to a target sequence found in the library such that the targeting oligonucleotide hybridizes to the target sequence, wherein hybridization of the targeting oligonucleotide to the target sequence forms a region of double-stranded DNA (dsDNA) that comprises a protospacer adjacent motif (PAM) and at least 12 nucleotides downstream of the PAM; (c) contacting the library with a nucleic acid-guided nuclease and a guide nucleic acid (gNA) comprising a region complementary to the targeting oligonucleotide or the target sequence, such that the gNA hybridizes with the targeting oligonucleotide or the target sequence and recruits the nuclease to cleave the target sequence; and (d) amplifying the library using primers that hybridize to the 5′ adapter and the 3′ adapter, thereby generating an amplified library in which the target sequence is depleted and the sequences of interest are enriched.

In Protocol 2 (see FIG. 1 at arrow 2), the method comprises: (a) contacting the library with a targeting oligonucleotide that is complementary to a target sequence found in the library such that the targeting oligonucleotide hybridizes to the target sequence, wherein hybridization of the targeting oligonucleotide to the target sequence forms a region of dsDNA that comprises a PAM and at least 12 nucleotides downstream of the PAM; (b) contacting the library with a nucleic acid-guided nuclease and a gNA comprising a region complementary to the targeting oligonucleotide or the target sequence, such that the gNA hybridizes with the targeting oligonucleotide or the target sequence and recruits the nuclease to cleave the target sequence; and (c) amplifying the library using primers that hybridize to the 5′ adapter and the 3′ adapter, thereby generating an amplified library in which the target sequence is depleted and the sequences of interest are enriched.

In Protocol 3 (see FIG. 1, arrow 3), the method comprises: (a) contacting the library with proteinase K, thereby degrading at least a portion of the SSBs; (b) incubating the library to allow the paired ssDNA molecule to hybridize to the target sequence, wherein hybridization of the paired ssDNA molecule to the target sequence forms a region of dsDNA that comprises a PAM and at least 12 nucleotides downstream of the PAM; (c) contacting the library with a nucleic acid-guided nuclease and a gNA comprising a region complementary to the paired ssDNA molecule or to the target sequence, such that the gNA hybridizes with the paired ssDNA molecule or the target sequence and recruits the nuclease to cleave the target sequence; and (d) amplifying the library using primers that hybridize to the 5′ adapter and the 3′ adapter, thereby generating an amplified library in which the target sequence is depleted and the sequences of interest are enriched.

In a second aspect, the present invention provides methods for depleting a target non-polyadenylated RNA (poly(A)⁻ RNA) molecule from a sample comprising single-stranded RNA (ssRNA) molecules (see FIG. 2). The methods comprise: (a) contacting the sample with a blocker oligonucleotide that comprises a 3′ portion that is complementary to a target sequence comprising the 3′ end of the target poly(A)⁻ RNA molecule, such that the 3′ portion of the blocker oligonucleotide hybridizes to the target sequence and a 5′ portion of the blocker oligonucleotide forms a single-stranded overhang; (b) contacting the sample with a poly A polymerase and ATP, thereby adding a poly(A) tail to the 3′ end of the ssRNA molecules that are not bound by the blocker oligonucleotide; (c) hybridizing a poly(dT) primer to the poly(A) tails; (d) reverse transcribing the ssRNA molecules bound by the poly(dT) primer to generate ssDNA molecules that comprise the poly(dT) primer on the 5′ end; (e) ligating an adapter to the 3′ end of the ssDNA molecules; and (f) amplifying the ssDNA molecules using amplification primers that hybridize to the poly(dT) primer and the adapter, thereby generating an amplified library in which the target poly(A)⁻ RNA molecule has been depleted.

### BRIEF DESCRIPTION OF THE DRAWINGS

FIG. 1 shows a schematic depiction of the methods disclosed herein for depleting an unwanted sequence from a single-stranded DNA (ssDNA) library. These methods are subdivided into three protocols (i.e., Protocol 1-3), which are labeled on the left. The schematic at the top of the page shows an exemplary ssDNA molecule (horizontal line) found in the library. The ssDNA molecule comprises adapters on the 5′ and 3′ ends (arrows), protospacer adjacent motif (PAM) sites for a nucleic-acid guided nuclease (rectangles), and single-stranded DNA binding proteins (SSBs; ovals). In Protocol 1, the library is contacted with proteinase K to degrade the SSBs. Then, a targeting oligonucleotide is hybridized to a target sequence to forms a region of double-stranded DNA (dsDNA) comprising the PAM and at least 12 nucleotides downstream of the PAM. In Protocol 2, the library is simply contacted with the targeting oligonucleotide in the absence of proteinase K. The targeting oligonucleotide anneals to the subset of the target sequences that are not occluded by SSBs. In Protocol 3, the library includes a paired ssDNA molecule that is complementary to a target sequence found in the library. Thus, the library is simply contacted with proteinase K to degrade the SSBs to allow the paired ssDNA molecule to bind to the target sequence. All three of the protocols involve two final steps (not shown): a nucleic acid-guided nuclease and a guide nucleic acid (gNA) are used to cleave the target sequences, and the library is amplified using primers that hybridize to the 5′ adapter and the 3′ adapter.

FIG. 2 shows a schematic depiction of the methods disclosed herein for depleting an unwanted non-polyadenylated RNA from a pool of single-stranded RNA molecules. The left side of the figure shows how sequences of interest (e.g., non-ribosomal RNA sequences) are processed, while the right side shows how the unwanted target sequences (e.g., ribosomal RNA sequences) are processed. In these methods, the sample is contacted with a blocker oligonucleotide that hybridizes to the unwanted target sequence and forms a single-stranded overhang. Then, the sample is contacted with a polyA polymerase and ATP, such that a poly(A) tail is added to the 3′ end of the ssRNA molecules that are not bound by the blocker oligonucleotide. Next, a poly(dT) primer is hybridized to the poly(A) tails and used to reverse transcribe ssDNA from the polyadenylated ssRNA molecules. Finally, an adapter is ligated to the 3′ end of the ssDNA molecules, and the ssDNA molecules are amplified using amplification primers that hybridize to the poly(dT) primer and the adapter (e.g., using the SRSLY protocol).

### DETAILED DESCRIPTION

The present invention provides methods for selectively depleting unwanted sequences from a pool of nucleic acids, including (1) methods that can be used to deplete an unwanted sequence from a single-stranded DNA library, and (2) methods that can be used to deplete an unwanted non-polyadenylated RNA from a pool of single-stranded RNA molecules. By depleting the unwanted sequences, the methods enrich a sample for sequences of interest. Thus, these methods can be used to generate enriched libraries for use in cloning, sequencing, and genotyping applications.

The term “depletion” refers to a process in which the amount of an unwanted sequence that is present in a pool of nucleic acids is reduced. In some cases, the unwanted sequence may be completely eliminated from the pool of nucleic acids. Depletion of an unwanted sequence “enriches” a sample for sequences of interest, i.e., it increases the amount or percentage of the sequences of interest in the sample relative to the amount of unwanted sequences.

As used herein, the term “sequences of interest” is used to refer all the sequences within a sample other than the unwanted sequence that are targeted for depletion. Examples of sequences of interest are provided below. The methods of the present invention may be used to enrich sequences of interest that are relatively scarce within a sample. For example, in some embodiments, the sequences of interest comprise less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.5%, less than 0.1%, less than 0.05%, less than 0.01%, less than 0.005%, or even less than 0.001% of the sample.

Methods for Depleting an Unwanted Species from a Single-Stranded DNA Library:

In a first aspect, the present invention provides methods for depleting an unwanted target sequence from a single-stranded DNA (ssDNA) library, thereby enriching for sequences of interest within the library. These methods can be subdivided into three protocols (i.e., Protocol 1, Protocol 2, and Protocol 3), which are each described below.

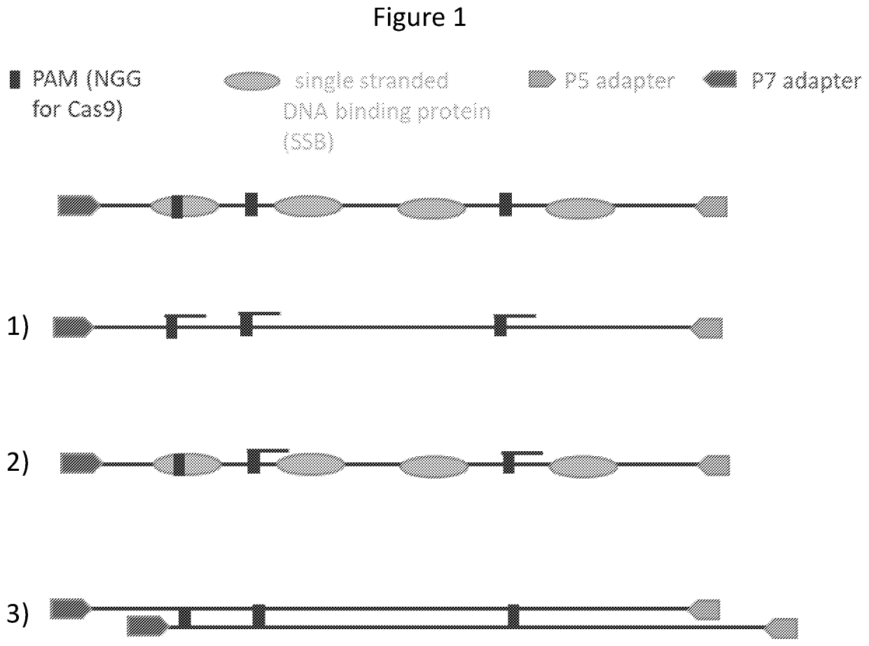

In these methods, cleavage by a nucleic acid-guided nuclease (e.g., Cas9) is used to prevent amplification of an unwanted target sequence. The library used in these methods is composed of ssDNA molecules that comprise adapters on both ends (i.e., a 5′ adapter and a 3′ adapter) and are bound by single-stranded DNA-binding proteins (SSBs). Thus, cleavage by a nucleic acid-guided nuclease generates ssDNA fragments that lack an adapter on one end (i.e., at the cut site). This allows the remaining, uncleaved ssDNA molecules (i.e., the sequences of interest) to be selectively amplified using primers that hybridize to the adapter sequences. Notably, since many nucleic acid-guided nucleases can only cut double-stranded DNA (dsDNA), these methods require that the unwanted ssDNA target sequence is converted into dsDNA via hybridization to a complementary sequence prior to nuclease cleavage (see FIG. 1).

As used herein, a “library” is a collection of DNA fragments. Any type of DNA library may be used with the present invention including, for example, cDNA libraries (formed from reverse-transcribed RNA), genomic libraries (formed from genomic DNA), and randomized mutant libraries (formed by the random incorporation of nucleotides in a de novo synthesis reaction). In general, a library is prepared by inserting DNA fragments from a particular source into cloning vectors and transferring the pool of recombinant vectors into a population of bacteria or yeast that are grown in culture to propagate the library. The library may be generated from DNA from any source including, without limitation, DNA found in a biological sample, clinical sample (e.g., blood, serum, plasma mucus, hair, urine, feces, saliva, breath, cerebrospinal fluid, lymph, tissue, skin, or a biopsy), forensic sample (e.g., a sample obtained from an individual at a crime scene or from a piece of evidence), environmental sample (e.g., soil, rock, plant, water, air), metagenomic sample, or food sample (e.g., meat, dairy, or produce). In some embodiments, the sample used to prepare the library is from a human. In some embodiments, the library is a library that was generated using a method for depleting non-polyadenylated RNA from a pool of single-stranded RNA molecules described in the following section.

The libraries used with the present methods comprise ssDNA molecules that include adapters on both ends (i.e., a 5′ adapter and a 3′ adapter). An “adapter” is a short (e.g., 10-100 nucleotide) oligonucleotide that is ligated to the end of another oligonucleotide. The adapters used with the present invention may be linear, Y-shaped, circular, or hairpin-shaped. The adapters can comprise multiple distinct sequences, such as a barcode, an index sequence, or a unique molecular identifier. In some embodiments, the adapters are sequencing adapters, i.e., adapters that comprise sequences that are designed to interact with a specific sequencing platform (e.g., the surface of a flow-cell) to facilitate a sequencing reaction.

As used herein, the terms “oligonucleotide,” “polynucleotide,” and “nucleic acid” are used interchangeably to refer to a polymer of DNA or RNA, which may be single-stranded or double-stranded, synthesized or obtained (e.g., isolated and/or purified) from natural sources, which may contain natural, non-natural or altered nucleotides, and which may contain natural, non-natural, or altered internucleotide linkages (e.g., a phosphoroamidate linkage or a phosphorothioate linkage).

ssDNA is vulnerable to chemical attack and nucleolytic degradation. Thus, the ssDNA libraries used with the present invention are bound by single-stranded DNA-binding proteins that serve to protect the libraries. The term “single-stranded DNA-binding protein (SSB)” refers to a protein that binds to and stabilizes single-stranded regions of DNA. Suitable SSBs include prokaryotic SSBs (e.g., bacterial or archaeal SSBs) and eukaryotic SSBs. Specific non-limiting examples of SSBs that may be used with the present inventions include E. coli SSB, E. coli RecA, Extreme Thermostable Single-Stranded DNA Binding Protein (ET SSB), Thermus thermophilus (Tth) RecA, T4 Gene 32 Protein, and replication protein A (RPA). Several of these proteins (i.e., ET SSB, Tth RecA, E. coli RecA, and T4 Gene 32 Protein) as well buffers and detailed protocols for preparing SSB-bound ssDNA are commercially available (e.g., from New England Biolabs, Inc).

Single-stranded DNA library preparation methods offer several advantages over traditional dsDNA methods. By denaturing the duplexed template DNA prior to adapter ligation and maintaining the DNA as single strands through at least an initial adapter ligation, single-stranded preparation methods are theoretically able to convert all the molecules captured by traditional dsDNA library preparation methods as well as nicked dsDNA and ssDNA molecules. Originally developed for the genomic analysis of highly degraded ancient DNA, ssDNA library preparation methods have been adopted for other fragmented sample types such as such as cell-free DNA (cfDNA) and DNA purified from formalin fixed paraffin embedded (FFPE) sections. ssDNA libraries are advantageous due to their efficiency in converting a high fraction of input DNA fragments into sequencing library molecules and their ability to capture small DNA fragments. Further, the sequencing reads from some ssDNA library methods represent the natural 5-prime and 3-prime ends of the input DNA fragments. Thus, when mapped to a reference genome, these data reveal the exact genomic location of the input fragments, which is an important feature for cfDNA researchers studying biological fragmentation patterns.

However, many nucleic acid-guided nucleases, including Cas9, require double-stranded DNA (dsDNA) and cannot bind to and cut ssDNA. Thus, available methods for Cas9-mediated depletion of target sequences can only be performed after the ssDNA has been converted into dsDNA by PCR. Post-PCR depletion is not ideal because Cas9 does not turn over or recycle and there are many more copies of the target sequences to be cut following PCR. In the methods of the present invention, this limitation is overcome by hybridizing a complementary sequence to the target oligonucleotide, thereby forming a region of dsDNA that can be bound and cut by a nucleic acid-guided nuclease prior to PCR. This allows Cas9-mediated depletion to be performed pre-PCR using a much lower amount of Cas9 than would be required to achieve the same level of depletion post-PCR.

In Protocol 1 (see FIG. 1, arrow 1), the method comprises: (a) contacting the library with proteinase K to degrade at least a portion of the SSBs; (b) contacting the library with a targeting oligonucleotide that is complementary to a target sequence found in the library such that the targeting oligonucleotide hybridizes to the target sequence, wherein hybridization of the targeting oligonucleotide to the target sequence forms a region of double-stranded DNA (dsDNA) that comprises a protospacer adjacent motif (PAM) and at least 12 nucleotides downstream of the PAM; (c) contacting the library with a nucleic acid-guided nuclease and a guide nucleic acid (gNA) comprising a region complementary to the targeting oligonucleotide or the target sequence, such that the gNA hybridizes with the targeting oligonucleotide or the target sequence and recruits the nuclease to cleave the target sequence; and (d) amplifying the library using primers that hybridize to the 5′ adapter and the 3′ adapter, thereby generating an amplified library in which the target sequence is depleted and the sequences of interest are enriched.

Protocol 2 (see FIG. 1, arrow 2) differs from Protocol 1 in that it skips the first proteinase K degradation step. Rather than degrade the SSBs bound to the ssDNA library, this protocol relies on the fact that a subset of the target sequences will not be occluded by SSBs. Specifically, in Protocol 2, the method comprises: (a) contacting the library with a targeting oligonucleotide that is complementary to a target sequence found in the library such that the targeting oligonucleotide hybridizes to the target sequence, wherein hybridization of the targeting oligonucleotide to the target sequence forms a region of dsDNA that comprises a PAM and at least 12 nucleotides downstream of the PAM; (b) contacting the library with a nucleic acid-guided nuclease and a gNA comprising a region complementary to the targeting oligonucleotide or the target sequence, such that the gNA hybridizes with the targeting oligonucleotide or the target sequence and recruits the nuclease to cleave the target sequence; and (c) amplifying the library using primers that hybridize to the 5′ adapter and the 3′ adapter, thereby generating an amplified library in which the target sequence is depleted and the sequences of interest are enriched.

In some embodiments, Protocol 2 is performed on a library that contains a reduced amount of SSBs. Use of such a library reduces the likelihood that the SSBs will interfere with the binding and function of the nucleic acid-guided nuclease. The SSB content of the library may be reduced by 20%, 30%, 40%, 50%, 60%, 70%, 80% or even as much as 90%, compared to a library that is fully saturated with SSBs, to produce a library with accessible target sequences and PAM sites. The SSB content of the library can be reduced by simply adding fewer SSBs to the library.

Protocol 3 (see FIG. 1, arrow 3) differs from Protocol 1 in that the library used in this protocol includes a paired ssDNA molecule comprising a portion that is complementary to a target sequence found in the library. Thus, the library already includes the necessary sequences to form a region of dsDNA comprising the target sequence, and a targeting oligonucleotide is not required. Specifically, in Protocol 3, the method comprises: (a) contacting the library with proteinase K, thereby degrading at least a portion of the SSBs; (b) incubating the library to allow the paired ssDNA molecule to hybridize to the target sequence, wherein hybridization of the paired ssDNA molecule to the target sequence forms a region of dsDNA that comprises a PAM and at least 12 nucleotides downstream of the PAM; (c) contacting the library with a nucleic acid-guided nuclease and a gNA comprising a region complementary to the paired ssDNA molecule or to the target sequence, such that the gNA hybridizes with the paired ssDNA molecule or the target sequence and recruits the nuclease to cleave the target sequence; and (d) amplifying the library using primers that hybridize to the 5′ adapter and the 3′ adapter, thereby generating an amplified library in which the target sequence is depleted and the sequences of interest are enriched.

Any of the above protocols can be used to deplete a plurality of target sequences, as opposed to a single target sequence, from a ssDNA library. For instance, in some embodiments of Protocol 1 or Protocol 2, the library is contacted with (i) a plurality of targeting oligonucleotides that are complementary to a plurality of target sequences found in the library, and (ii) a plurality of gNAs that are complementary to the plurality of targeting oligonucleotides or to the plurality of target sequences. Likewise, in some embodiments of Protocol 3, the library comprises a plurality of paired ssDNA molecules that are complementary to a plurality of target sequences, and step (c) comprises contacting the library with a plurality of gNAs that are complementary to the plurality of paired ssDNA molecules or to the plurality of target sequences. In these embodiments, the methods generate an amplified library in which the plurality of target sequences are depleted.

Without wishing to be bound by theory, the inventors suspect that the SSBs bound to the ssDNA library may sterically hinder cleavage by nucleic acid-guided nucleases. Thus, in Protocol 1 and Protocol 3, treatment with proteinase K is used to degrade at least a portion of the SSBs bound to the library. “Proteinase K” is a broad-spectrum serine protease that is commonly used to digest proteins. In contrast, Protocol 2 does not include a proteinase K treatment and instead relies on the fact that a subset of the target sequences will not be occluded by SSBs and that only one cut is required to deplete an unwanted ssDNA from the library.

In the present methods, a nucleic acid-guided nuclease is used to cleave the target sequence. The term “cleaving”, as used herein, refers to a reaction that breaks the phosphodiester bonds between two adjacent nucleotides in both strands of a double-stranded DNA molecule, thereby producing a double-stranded break in the DNA molecule. As used herein, a “nucleic acid-guided nuclease” is a nuclease that cleaves DNA, RNA, or DNA/RNA hybrids, and which uses one or more guide nucleic acids (gNAs) to confer specificity. Nucleic acid-guided nucleases include CRISPR/Cas system proteins as well as non-CRISPR/Cas system proteins. The nucleic acid-guided nuclease used with the present invention may be naturally occurring or engineered, and it may be isolated, recombinantly produced, or synthetic.

In some embodiments, the nucleic acid-guided nuclease is a CRISPR/Cas system protein. Suitable CRISPR/Cas system proteins include those from CRISPR Type I systems, CRISPR Type II systems, and CRISPR Type III systems. In some embodiments, the CRISPR/Cas system protein is selected from the group consisting of Cas9, CasX, CasY, Cpf1, Cas3, Cas8a-c, Cas10, Cse1, Csy1, Csn2, Cas4, Csm2, and Cm5. In some embodiments, the CRISPR/Cas system protein is Cas9.

The nucleic acid-guided nuclease may be from any bacterial or archaeal species. For example, in some embodiments, the nucleic acid-guided nuclease is from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, or Corynebacter diphtheria.

Nucleic acid-guided nucleases are recruited to a target site by a guide nucleic acid (gNA). As used herein, the term “target site” refers to a region of dsDNA comprising the target sequence hybridized to either the targeting oligonucleotide (in Protocol 1 and Protocol 2) or the paired ssDNA molecule (in Protocol 3). The target site is immediately adjacent to a protospacer adjacent motif (PAM), allowing it to be cleaved by the nucleic acid-guided nuclease. The gNAs used with the present invention selectively bind to a target site in the unwanted target sequence, and do not bind to the sequences of interest present in the library.

The gNA comprises a region that binds to the nucleic acid-guided nuclease (e.g., a tracrRNA), thereby forming a nucleic acid-guided nuclease-gNA complex. The gNA also comprises a “targeting region”, i.e., a region that is complementary to the target site (e.g., a crRNA). Hybridization of the gNA targeting region to the target site localizes the nucleic acid-guided nuclease to that site. The gNA targeting region may be complementary to either strand that forms the target site, i.e., either the target sequence or the targeting oligonucleotide/paired ssDNA molecule. The gNA targeting region may be 15, 16, 17, 18, 19, 20, 25, 30, 35, 40, 45, or 50 nucleotides in length. In some embodiments, the gNA is composed of two molecules that base pair to form a functional gNA: one comprising the region that binds to the nucleic acid-guided nuclease and one comprising a targeting region that binds to the target site. Alternatively, the gNA may be a single molecule comprising both of these components. In some embodiments, the gNA is a guide RNA (gRNA).

As used herein, the term “complementary” refers to the ability of a nucleic acid molecule to bind to (i.e., hybridize with) another nucleic acid molecule through the formation of hydrogen bonds between specific nucleotides (i.e., A with T or U and G with C), forming a double-stranded molecule. The term “hybridization” refers to the process by which a single-stranded oligonucleotide binds to a complementary strand through base pairing. A nucleic acid is considered to “selectively bind” to another nucleic acid sequence if the two sequences specifically hybridize to one another under moderate to high stringency hybridization and wash conditions, which are known in the art (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.).

Many nucleic acid-guided nucleases require dsDNA to cleave a target site. Thus, in the present methods, a target sequence within an unwanted ssDNA species is made double-stranded to allow the nucleic acid-guided nuclease to cut it. This is accomplished by hybridizing a complementary sequence to the target sequence. The complementary sequence used in Protocol 1 and Protocol 2 is referred to as a “targeting oligonucleotide”, whereas the complementary sequence used in Protocol 3 is referred to as a “paired ssDNA molecule”. The difference between these two types of complementary sequences is that a paired ssDNA molecule is an ssDNA molecule that is already present in the ssDNA library and does not need to be added, whereas the targeting oligonucleotide is not present in the ssDNA library and must be added.

Additionally, nucleic acid-guided nucleases require a protospacer adjacent motif (PAM) to cleave a target site. A “protospacer adjacent motif (PAM)” is a short DNA sequence (usually 2-6 base pairs in length) immediately adjacent to a target site that is recognized by the nucleic acid-guided nuclease. In the present methods, the region of dsDNA formed by hybridization of the targeting oligonucleotide/paired ssDNA molecule to the target sequence must comprise a PAM and sequence adjacent to the PAM. For example, to cut a target site, Cas9 requires that the PAM and at least 12 nucleotides downstream of the PAM are double-stranded. Different nucleic acid-guided nucleases recognize different PAM sequences. Exemplary PAM sequences recognized by Type II CRISPR system proteins include those from Streptococcus pyogenes (NGG), Staphylococcus aureus (NNGRRT), Neisseria meningitidis (NNNNGATT), Streptococcus thermophilus (NNAGAA), and Treponema denticola (NAAAAC). Accordingly, the gNA and target sequence should be designed/selected with a specific nucleic acid-guided nuclease in mind. The PAM may be present in either strand that forms the target site, i.e., either the target sequence or the targeting oligonucleotide/paired ssDNA molecule.

In the final step of the present methods, the library is amplified using primers that hybridize to the 5′ adapter and the 3′ adapter present in the ssDNA molecules. This ensures that the uncleaved sequences of interest are amplified, whereas the cleaved target sequences (which have at least one end that does not comprise an adapter) are not. The term “amplifying”, as used herein, refers to process by which one or more copies of a nucleic acid are produced using the nucleic acid as a template. Amplification may be exponential or linear. Common methods of amplification include polymerase chain reaction (PCR)-based methods, isothermal methods, and rolling circle methods. Additional amplification methods include, without limitation, ligase chain reaction (LCR), transcription-based amplification system (TAS), nucleic acid sequence-based amplification (NASBA), self-sustained sequence replication (3SR), strand displacement amplification (SDA), boomerang DNA amplification (BDA), and Q-beta replication.

As used herein, the terms “primer” and “amplification primer” refer to a short, single-stranded oligonucleotide that is complementary to (and can, therefore, hybridize to) a specific sequence. Primers serve as a site from which DNA synthesis can be initiated. Typically, in an amplification reaction, an upstream or “forward” primer and a downstream or “reverse” primer are used to delimit the region of the nucleic acid to be amplified. The primers used with the present methods comprise a portion that is complementary to the 5′ adapter or the 3′ adapter. The primers may further comprise additional sequences, such as sequences that are useful for next-generation sequences (e.g., index or barcode sequences).

The purpose of the present methods is to deplete one or more target sequences from an ssDNA library, thereby enriching the library for sequences of interest. A “target sequence” can be any known, unwanted ssDNA sequence. For example, the methods may be used to deplete a target sequence that is of little value for a downstream application. For sequencing applications, this ensures that fewer sequencing reads are wasted on the target sequence and that greater sequencing depth and coverage will be achieved for the sequences of interest. Suitable target sequences include nucleic acids of little informative value, such as mitochondrial DNA, repetitive sequences, multi-copy sequences, sequences encoding globin proteins, sequences encoding a transposon, sequences encoding retroviral sequences, sequences comprising telomere sequences, sequences comprising sub-telomeric repeats, sequences comprising centromeric sequences, sequences comprising intron sequences, sequences comprising Alu repeats, SINE repeats, LINE repeats, dinucleic acid repeats, trinucleic acid repeats, tetranucleic acid repeats, poly-A repeats, poly-T repeats, poly-C repeats, poly-G repeats, AT-rich sequences, and GC-rich sequences.

For example, when one is interested in sequences found within the nuclear genome, it can be advantageous to remove mitochondrial DNA from the library. Thus, in some embodiments, the sequences of interest are from nuclear DNA (i.e., DNA found within the nucleus of a eukaryotic cell), and the target sequence(s) are from mitochondrial DNA. In other cases, when one is interested in detecting a pathogen within a host organism, it can be advantageous to remove host organism DNA from the library. Thus, in some embodiments, the sequences of interest are from a pathogen, and the target sequence(s) are from the host organism. As used herein, the term “pathogen” refers to a disease-causing microorganism (e.g., a bacteria, virus, fungus, algae, or protozoan) and the term “host organism” refers to the organism that the pathogen has infected. In other cases, when one is interested in detecting genetic mutations found in tumors, it can be advantageous to remove DNA from “normal” (i.e., wild-type) cells. Thus, in some embodiments, the sequences of interest are from a mutant tumor cell, and the target sequence(s) are from wild-type cells. In other cases, when one is interested in studying a rare species or assessing the diversity within a microbiome, it can be advantageous to remove DNA from a particularly abundant species that populates that microbiome. Thus, in some embodiments, the sequences of interest are from a microbiome, and the target sequence(s) are from a particularly abundant species found in the microbiome.

Methods for Depleting Non-Polyadenylated RNA from a Pool of Single-Stranded RNA Molecules:

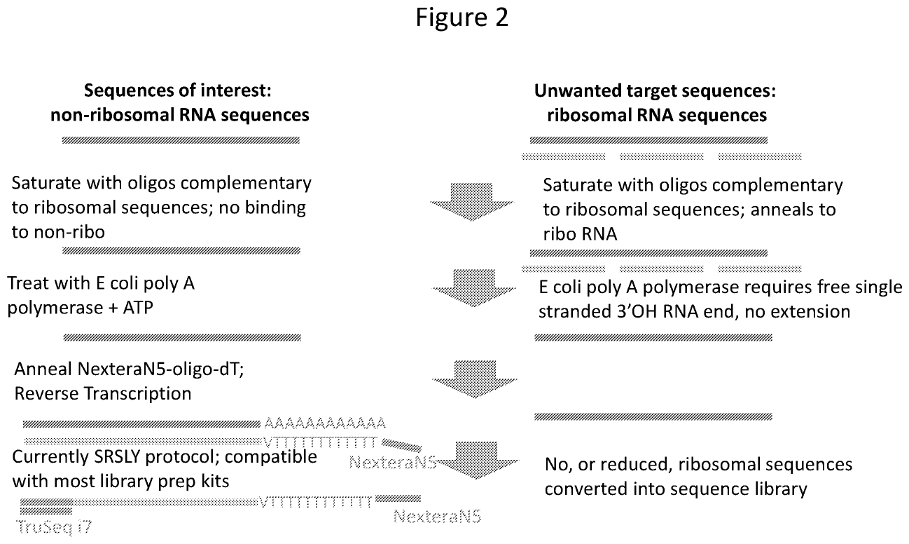

In a second aspect, the present invention provides methods for depleting a target non-polyadenylated RNA (poly(A)⁻ RNA) molecule from a sample comprising single-stranded RNA (ssRNA) molecules. In these methods, a blocker oligonucleotide is selectively bound to a target sequence, which blocks the target sequence from being polyadenylated by a poly(A) polymerase. This allows the remaining sequences of interest to be polyadenylated and selectively amplified (See FIG. 2).

In traditional methods for depleting rRNA, labeled (e.g., biotinylated) probes are hybridized to rRNA-specific sequences and the label is used to capture and remove the bound rRNAs (e.g., using magnetic streptavidin beads). In contrast, the methods of the present invention do not require separate depletion and capture steps. Thus, the disclosed methods are expected to be easier, cheaper, faster, and require less hands-on time than the methods of the prior art.

Specifically, the methods comprise: (a) contacting the sample with a blocker oligonucleotide that comprises a 3′ portion that is complementary to a target sequence comprising the 3′ end of the target poly(A)⁻ RNA molecule, such that the 3′ portion of the blocker oligonucleotide hybridizes to the target sequence and a 5′ portion of the blocker oligonucleotide forms a single-stranded overhang; (b) contacting the sample with a polyA polymerase and ATP, thereby adding a poly(A) tail to the 3′ end of the ssRNA molecules that are not bound by the blocker oligonucleotide; (c) hybridizing a poly(dT) primer to the poly(A) tails; (d) reverse transcribing the ssRNA molecules bound by the poly(dT) primer to generate ssDNA molecules that comprise the poly(dT) primer on the 5′ end; (e) ligating an adapter to the 3′ end of the ssDNA molecules; and (f) amplifying the ssDNA molecules using amplification primers that hybridize to the poly(dT) primer and the adapter, thereby generating an amplified library in which the target poly(A)⁻ RNA molecule has been depleted.

These methods can be used to deplete a plurality of target poly(A)⁻ RNA molecules as opposed to a single target poly(A)⁻ RNA molecule. For example, in some embodiments, step (a) comprises contacting the library with a plurality of blocker oligonucleotides that comprise 3′ portions that are complementary to a plurality of target sequences comprising the 3′ ends of a plurality of target poly(A)⁻ RNA molecules, such that the method generates an amplified library in which the plurality of target poly(A)⁻ RNA molecules has been depleted.

As used herein, the term “non-polyadenylated RNA (poly(A)⁻ RNA)” refers to an RNA molecule that lacks a poly(A) tail on the 3′ end. While a poly(A) tail is added post-transcriptionally to the 3′ end of almost all eukaryotic messenger RNAs (mRNAs), a number of functional RNAs are known to lack poly(A) tails. Known poly(A)⁻ RNAs include ribosomal RNAs (rRNAs) generated by RNA polymerase I and III, other small RNAs generated by RNA polymerase III, replication-dependent histone mRNAs, and a few long non-coding RNAs (lncRNAs) synthesized by RNA polymerase II. A “target poly(A)⁻ RNA” can be any known, unwanted poly(A)⁻ RNA. In some embodiments, the target poly(A)⁻ RNA is a ribosomal RNA (rRNA). In these embodiments, the target sequence may be specific to rRNA, meaning that it is present in rRNAs but not in other classes of RNA.

The methods for depleting a target poly(A)⁻ RNA described herein can be performed on any sample comprising single-stranded RNA (ssRNA) molecules. Suitable samples include, but are not limited to, biological samples, clinical samples (e.g., blood, serum, plasma mucus, hair, urine, feces, saliva, breath, cerebrospinal fluid, lymph, tissue, skin, or a biopsy), forensic samples (e.g., a sample obtained from an individual at a crime scene or from a piece of evidence), environmental samples (e.g., soil, rock, plant, water, air), metagenomic samples, food samples (e.g., meat, dairy, or produce), and the like. In some embodiments, the sample has been processed (e.g., to isolate, shear, and/or amplify the ssRNA). In other embodiments, the sample is unprocessed.

“Polyadenylation” is the addition of a poly(A) tail to an RNA molecule. A “poly(A) tail” consists of multiple adenosine monophosphates. In other words, it is a stretch of RNA that comprises only adenine bases. Polyadenylation is accomplished using a “polyA polymerase,” an enzyme that catalyzes the template independent addition of adenosine monophosphate (AMP) from adenosine triphosphate (ATP) to the 3′ end of an RNA molecule. Importantly, poly A polymerase can only add AMP to a free, single stranded 3′OH. Thus, this enzyme will not polyadenylate RNA molecules in which the 3′OH has been blocked (e.g., by a 5′ overhang), but it will polyadenylate RNA molecules in which the 3′OH remains accessible. In some embodiments, the poly A polymerase is from E. coli.

In the present methods, the 3′ end of the target poly(A)⁻ RNA is blocked from being polyadenylated using a blocker oligonucleotide. A “blocker oligonucleotide” is an oligonucleotide that comprises a 3′ portion that is complementary to a “target sequence” within the target poly(A)⁻ RNA. The target sequence comprises the 3′ end of the target poly(A)⁻ RNA molecule, such that the 3′ portion of the blocker oligonucleotide hybridizes to the target sequence and a 5′ portion of the blocker oligonucleotide forms a single-stranded overhang. The blocker oligonucleotide should be designed to hybridize to a target sequence that is specific to the target poly(A)⁻ RNA, such that the blocker oligonucleotide only binds to and blocks the polyadenylation of the target poly(A)⁻ RNA and the remaining ssRNA molecules (i.e., the sequences of interest) remain accessible for polyadenylation.

The term “poly(dT) primer” is used to refer to a primer comprising a poly(dT) sequence. The poly(dT) portion of this primer is complementary to a poly(A) tail. Thus, the poly(dT) primer selectively binds to ssRNA molecules that have been polyadenylated. The poly(dT) primer may further comprise additional sequences, including sequences that are useful for next-generation sequences, e.g., sequencing adapters, index sequences, and barcode sequences.

After it is hybridized to the polyadenylated ssRNA molecules (i.e., the sequences of interest), the poly(dT) primer is used to reverse transcribe the bound ssRNA molecules into ssDNA molecules that comprise the poly(dT) primer on the 5′ end. “Reverse transcription” is a process in which an enzyme (i.e., a reverse transcriptase) is used to generate complementary DNA (cDNA) from an RNA template. In this process, reverse transcriptase adds deoxynucleotides (dNTPs) to extend a primer that is bound to the RNA. Methods for performing reverse transcription are well known in the art.

To prepare the ssDNA molecules (i.e., cDNA) produced by the reverse transcription reaction for amplification, an adapter is ligated to the 3′ end of the molecules. This allows the ssDNA molecules generated from the sequences interest to be amplified using amplification primers that hybridize to the poly(dT) primer and the adapter. As used herein, the term “ligating” refers to an enzymatically catalyzed process by which the terminal nucleotide at the 5′ end of a first DNA molecule is joined to the terminal nucleotide at the 3′ end of a second DNA molecule. Ligation is commonly performed using the enzyme T4 DNA ligase, which catalyzes the formation of covalent phosphodiester linkages between two DNA molecules. In some embodiments, ligation is performed using the Single Reaction Single-stranded LibrarY (SRSLY) method, in which ssDNA molecules are phosphorylated and SRSLY splint adapters are ligated to the ssDNA molecules in a combined phosphorylation/ligation reaction. For a detailed description of the SRSLY method, see BMC Genomics (2019) 20(1): 1023, which is hereby incorporated by reference in its entirety.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method of enriching for sequences of interest within a library, wherein the library comprises single-stranded DNA (ssDNA) molecules that each comprise a 5′ adapter and a 3′ adapter and are bound by single-stranded DNA-binding proteins (SSBs), the method comprising:
a) contacting the library with proteinase K to degrade at least a portion of the SSBs;
b) contacting the library with a targeting oligonucleotide that is complementary to a target sequence found in the library such that the targeting oligonucleotide hybridizes to the target sequence, wherein hybridization of the targeting oligonucleotide to the target sequence forms a region of double-stranded DNA (dsDNA) that comprises a protospacer adjacent motif (PAM) and at least 12 nucleotides downstream of the PAM;
c) contacting the library with a nucleic acid-guided nuclease and a guide nucleic acid (gNA) comprising a region complementary to the targeting oligonucleotide or the target sequence, such that the gNA hybridizes with the targeting oligonucleotide or the target sequence and recruits the nuclease to cleave the target sequence; and
d) amplifying the library using primers that hybridize to the 5′ adapter and the 3′ adapter, thereby generating an amplified library in which the target sequence is depleted and the sequences of interest are enriched.

2. (canceled)

3. (canceled)

4. The method of claim 1, wherein the library is contacted with:
i. a plurality of targeting oligonucleotides that are complementary to a plurality of target sequences found in the library, and
ii. a plurality of gNAs that are complementary to the plurality of targeting oligonucleotides or to the plurality of target sequences,
such that the method generates an amplified library in which the plurality of target sequences are depleted.

5. A method of enriching for sequences of interest within a library, wherein the library comprises ssDNA molecules that each comprise a 5′ adapter and a 3′ adapter and are bound by SSBs, and wherein the library comprises a paired ssDNA molecule comprising a portion that is complementary to a target sequence found in the library, the method comprising:
a) contacting the library with proteinase K, thereby degrading at least a portion of the SSBs;
b) incubating the library to allow the paired ssDNA molecule to hybridize to the target sequence, wherein hybridization of the paired ssDNA molecule to the target sequence forms a region of dsDNA that comprises a PAM and at least 12 nucleotides downstream of the PAM;
c) contacting the library with a nucleic acid-guided nuclease and a gNA comprising a region complementary to the paired ssDNA molecule or to the target sequence, such that the gNA hybridizes with the paired ssDNA molecule or the target sequence and recruits the nuclease to cleave the target sequence; and
d) amplifying the library using primers that hybridize to the 5′ adapter and the 3′ adapter, thereby generating an amplified library in which the target sequence is depleted and the sequences of interest are enriched.

6. The method of claim 5, wherein the library comprises a plurality of paired ssDNA molecules that are complementary to a plurality of target sequences, and wherein step (c) comprises contacting the library with a plurality of gNAs that are complementary to the plurality of paired ssDNA molecules or to the plurality of target sequences, such that the method generates an amplified library in which the plurality of target sequences are depleted.

7. The method of claim 1, wherein the nucleic acid-guided nuclease is a CRISPR/Cas system protein.

8. The method of claim 7, wherein the CRISPR/Cas system protein is selected from the group consisting of Cas9, CasX, CasY, Cpf1, Cas3, Cas8a-c, Cas10, Cse1, Csy1, Csn2, Cas4, Csm2, and Cm5.

9. The method of claim 8, wherein the CRISPR/Cas system protein is Cas9.

10. The method of claim 1, wherein the gNA is a guide RNA.

11. The method of claim 1, wherein the library was generated from a sample selected from the group consisting of a biological sample, a clinical sample, a forensic sample, and an environmental sample.

12. The method of claim 11, wherein the sample is from a human.

13. The method of claim 1, wherein the sequences of interest are from nuclear DNA.

14. The method of claim 1, wherein the sequences of interest are from a pathogen.

15. The method of claim 1, wherein the sequences of interest are from a mutant tumor cell.

16. The method of claim 1, wherein the sequences of interest are from a microbiome.

17. A method for depleting a target non-polyadenylated RNA (poly(A)⁻ RNA) molecule from a sample comprising single-stranded RNA (ssRNA) molecules, the method comprising:
a) contacting the sample with a blocker oligonucleotide that comprises a 3′ portion that is complementary to a target sequence comprising the 3′ end of the target poly(A)⁻ RNA molecule, such that the 3′ portion of the blocker oligonucleotide hybridizes to the target sequence and a 5′ portion of the blocker oligonucleotide forms a single-stranded overhang;
b) contacting the sample with a polyA polymerase and adenosine triphosphate (ATP), thereby adding a poly(A) tail to the 3′ end of the ssRNA molecules that are not bound by the blocker oligonucleotide;
c) hybridizing a poly(dT) primer to the poly(A) tails;
d) reverse transcribing the ssRNA molecules bound by the poly(dT) primer to generate ssDNA molecules that comprise the poly(dT) primer on the 5′ end;
e) ligating an adapter to the 3′ end of the ssDNA molecules; and
f) amplifying the ssDNA molecules using amplification primers that hybridize to the poly(dT) primer and the adapter, thereby generating an amplified library in which the target poly(A)⁻ RNA molecule has been depleted.

18. The method of claim 17, wherein step (a) comprises contacting the library with a plurality of blocker oligonucleotides that comprise 3′ portions that are complementary to a plurality of target sequences comprising the 3′ ends of a plurality of target poly(A)⁻ RNA molecules, such that the method generates an amplified library in which the plurality of target poly(A)⁻ RNA molecules has been depleted.

19. The method of claim 17, wherein the target sequence is specific to the target poly(A)⁻ RNA.

20. The method of claim 17, wherein the target poly(A)⁻ RNA is a ribosomal RNA (rRNA).

21. The method of claim 17, wherein the polyA polymerase is from E. coli.

22. The method of claim 17, wherein step (e) is performed using the Single Reaction Single-stranded LibrarY (SRSLY) method.

23. (canceled)

24. The method of claim 1, wherein the amplified library is used for cloning, sequencing, or genotyping.
